(19) United States
(12) Patent Application Publication
(10) Pub. No.: US 2005/0032230 A1
(43) Pub. Date: Feb. 10, 2005

(54) Method and apparatus for analyzing gas for trace amounts of oxygen

(75) Inventor: Pasl Abdel Jalil, Khobar (SA)

Correspondence Address: DENNISON, SCHULTZ & DOUGHERTY, 612 Crystal Square 4, 1745 Jefferson Davis Highway, Arlington, VA, 22202-3417, US

(73) Assignee: KING FAHD UNIVERSITY OF PETROLEUM AND MINERALS

(21) Appl. No.: 10/636,807

(22) Filed: Aug. 8, 2003

(51) Int. Cl. G01N 33/00

(52) U.S. Cl. 436/138

(57) ABSTRACT

A method for detecting ultra low trace amounts of oxygen in a relatively pure gas with a gas chromatograph and flame ionization detector is disclosed. The method includes the step of separating the oxygen from the other gas using a gas chromatograph. The separated gas is then converted to a carbon oxide or carbon oxides by passing the oxygen over a heated carbon material. The carbon oxides are then converted to methane by mixing the carbon oxides with hydrogen in the presence of a heated nickel catalyst. The methane is then introduced into a flame ionization detector which indicates a count indicative of the amount of methane. The methane count is indicative of the amount of oxygen in the original sample.

### FIELD OF THE INVENTION

This invention relates to a method and apparatus for analyzing a gas for trace amounts of oxygen and more particularly to a method and apparatus for analyzing trace amounts of oxygen in a relatively pure gas using gas chromatography and a flame ionization detector.

### BACKGROUND OF THE INVENTION

For many years, the gas industry has been faced with ever more stringent requirements for purity in industrial gases for research and for the electronic fabrication industries. For example, the need for higher purity was recognized in U.S. Pat. No. 5,612,489 of Ragsdale et al. As recognized in the Ragsdale et al. patent, oxygen is one of the contaminant gases for whichever more stringent requirements are needed particularly for inert gases used for prevention of oxidation. Typical gases include nitrogen and argon. As taught by Ragsdale et al. a low level of interactive gas is doped into the carrier gas carrying the sample gas which is to be analyzed with a known low level typically less then 10 parts per million (PPM) of doping oxygen into the carrier gas. By so doing, the detection limit for the trace oxygen is improved. The patent states that this technique permits reproducible detection of trace oxygen at quantities less than 1000 ppm. It also alleges that it is useful in the range of less then 1 ppm on a volumetric basis. However, in such systems, a detector that is sensitive to the interactive gas i.e. oxygen must be used.

As stated in Ragsdale et al. a detector sensitive to the interactive gas is selected from the group consisting of a thermo conductivity detector, a discharge ionization detector, a helium ionization detector and a high frequency discharged detector.

Conventional gas chromatography cannot reach the low detection levels of oxygen required if a thermal conductivity detector is used. To be more specific, thermal conductivity detectors do not achieve even low parts ppm range for oxygen molecules. Further, flame ionization detectors had not been used as oxygen detectors, because they use oxygen as an oxidizing agent in the flame.

It is now believed that there is a commercial market for an improved method and apparatus for detecting trace amounts of oxygen in a relatively pure inert gas using gas chromatography and a flame ionization detector. There should be a demand because it has been found that such apparatus and methods are capable of detecting trace amounts of oxygen in amounts of less than 560 ppb oxygen with a conventional gas chromatograph and a flame ionization detector.

### BRIEF SUMMARY OF THE INVENTION

In essence, the present invention contemplates an improved method for analyzing trace amounts of oxygen in a gas mixture such as a relatively pure gas. The oxygen is separated from the gas mixture as for example in a gas chromatograph and the separated oxygen gas is converted to a carbon oxide such as carbon monoxide and/or carbon dioxide by passing the oxygen over carbon at an elevated temperature. The carbon oxides are then converted to methane by mixing the carbon oxides with hydrogen with a heated nickel catalyst. The methane is then introduced into a flame ionization detector which produces a count indicative of the amount of methane. This amount of methane is indicative of the amount of oxygen in the original gas mixture.

In a preferred embodiment of the invention, the method for analyzing trace amounts of oxygen in a gas mixture includes the step of comparing the amount of methane produced in a control sample with the amount of methane produced in a test sample as an indication of the amount of oxygen in the test sample.

The preferred embodiment of the invention also includes the steps of providing a gas chromatograph, a flame ionization detector, a mass of heated carbon material and a heated nickel catalyst. In this embodiment of the invention, a gas to be analyzed such as a relatively pure gas with a suspected trace amount of oxygen is mixed with a carrier gas such as helium and introduced into a gas chromatograph . The carrier gas and relatively pure gas mixture is passed through a column of a gas chromatograph to separate any oxygen from the relatively pure gas. The oxygen and carrier gas is then passed through a mass of carbon at an elevated temperature to thereby form carbon oxides i.e. carbon monoxide and/or carbon dioxide. The carbon oxide and a mass of hydrogen gas is then passed over a heated nickel catalyst to form methane which is then introduced into a flame ionization detector. The detector provides an indication of the amount of methane and consequently an indication of an amount of oxygen in the relatively pure gas which is being tested.

### DESCRIPTION OF THE DRAWINGS

FIG. 1 is a schematic illustration of a conventional gas chromatograph;

FIG. 2 is a schematic illustration of a conventional injector port for use with a conventional gas chromatograph as used in the present invention;

FIG. 3 is a schematic illustration of a device for analyzing trace amounts of oxygen in accordance with the present invention;

FIG. 4 is a trace illustrating the base line of the flame ionization detector without injection of any sample;

FIG. 5a is a graph taken from a strip chart recorder illustrating periodic injections of oxygen having 520 ppb standard gas, with retention times of 2.67, 7.66, 12.67, etc.

FIG. 5b is the second run of injections of 520 ppb standard gas with the same retention times as FIG. 5a.

### DESCRIPTION OF THE PREFERRED EMBODIMENTS OF THE INVENTION

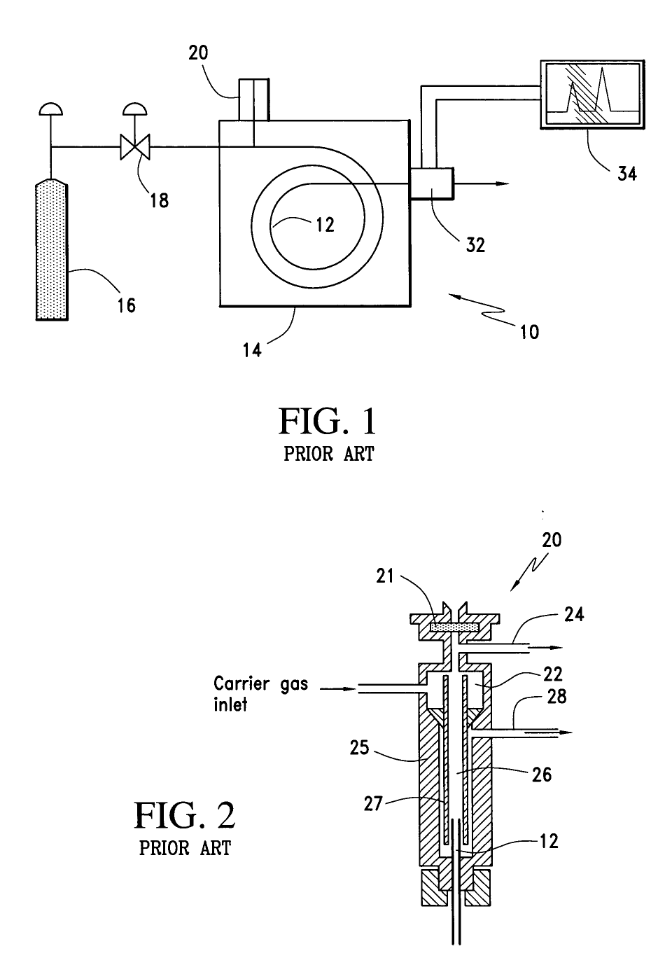

FIG. 1 is a schematic diagram of a conventional gas chromatograph 10 which includes a column 12 disposed in a column oven 14. An inert carrier gas such as nitrogen, helium or argon passes through a flow controller 18 and into the column 12. An injector port 20 is used to introduce a sample gas into the column 12.

An example of a conventional injector port 20 is illustrated in FIG. 2 wherein a rubber septum 21 is provided for injecting a gas sample into a chamber 22 for mixing with the carrier gas. A septum purge outlet 24 is provided between the septum 21 and chamber 22 and prevents bleed components from entering the chamber 22.

As illustrated in FIG. 2, the carrier gas and sample gas pass through a second chamber 26 which is disposed within a heated metal block 25 and glass liner 27 and into column 12. As illustrated, the injector port may also include a split outlet 28.

The carrier gas and sample gas pass through the column 12 and into a conventional detector 32 which indicates the composition of the sample gas and records the composition with a conventional recorder 34.

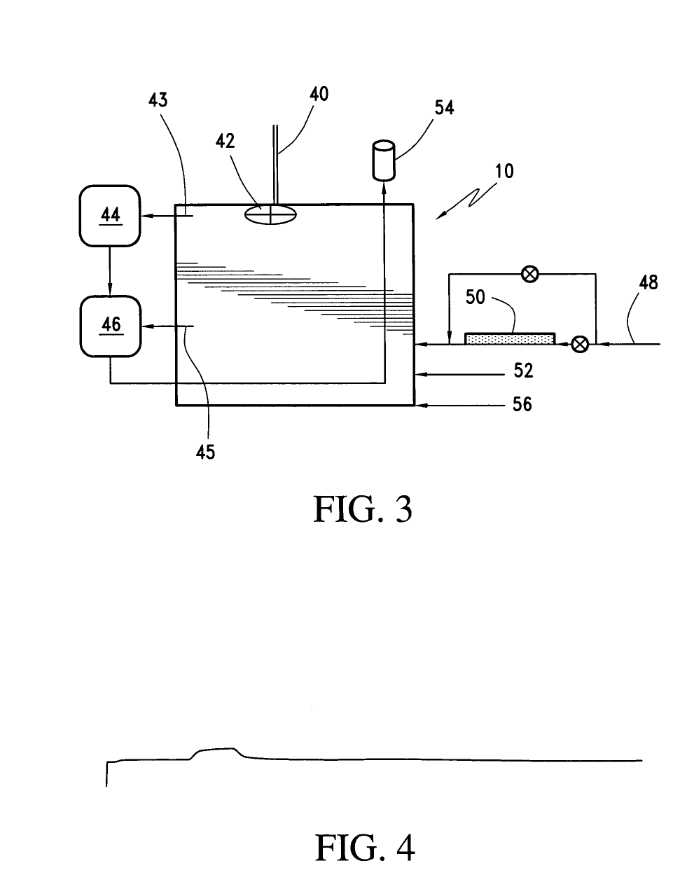

In the practice of the present invention, an inlet 40 shown in FIG. 3 is used to inject a sample of a gas to be analyzed into a gas chromatograph 10. The inlet 40 may also be provided with an automatic sampling valve 42 which is connected to a sampling loop (not shown) which in turn is connected to the column (not shown in FIG. 3) inside of the gas chromatograph 10. As illustrated, the outlet of the column indicated by the arrow 43 passes into a heated carbon containing tube 44 where the separated oxygen is converted into carbon oxides such as carbon monoxide and carbon dioxide.

The heated carbon may comprise most clean carbon sources. For example, charcoal or activated carbon can be used. The carbosieve used in the examples, disclosed herein, was taken from a charcoal filter from a Chrompack International (catalogue number 07942) of Middelburg, Netherlands. The particle size used ranged from 1-2 mm. The temperature used was 540° C. but could be higher.

Hydrogen gas is fed to a chamber 46 from the gas chromatograph as indicated by an arrow 45. The chamber 46 contains a heated nickel catalyst which converts the carbon oxides from the heated carbon containing tube 44 into methane.

As illustrated in FIG. 3, a carrier gas 48 such as helium is introduced through a Hewlett Packard oxygen scrubber 50 to less then 1 ppm into the gas chromatograph 10 where it is mixed with the inlet gas to be analyzed before passing through a column (not shown in FIG. 3) for separating the oxygen from the relatively pure gas in the sample. As airline 52 for a flame ionization detector 54 and a hydrogen gas line 56 for the flame ionization detector and gas chromatograph are also provided.

FIG. 4 shows the base line of the flame ionization detector without any injection of a sample. The hump shown is due to by passing the oxygen scrubber 50 shown in FIG. 3.

Analyzing a relatively pure gas for trace amounts of oxygen using gas chromatography and a flame ionization detector will now be described with reference to FIG. 3. A relatively pure gas to be analyzed passes through inlet 40. The gas flow rate may be controlled and monitored precisely by a digital mass flow control/meter such as a Matheson, model 8274 controller/meter interfaced with a Matheson sensor-transducer model 8272-0413 for helium based gases and a Matheson sensor-transducer model 8272-0432 for nitrogen based gases. Inlet 40 may be heated if there is probable condensation of any component in the inlet 40. The gas from the inlet 40 is used to flush the sampling loop that is connected to the automatic sampling valve 42 in FIG. 3.

The temperature in the thermostat zones (e.g. sampling loop, nickel catalyst, injector, oven and detectors were controlled using the control system of an HP 5880A gas chromatograph. The carrier gas which was helium in the experiments bypasses the sampling loop before injection of the sample. Upon injection, the sample is mixed with the carrier gas and enters a column (not shown in FIG. 3) inside the gas chromatograph's oven.

The oxygen is separated from the other gases by the end of the column. The outlet of the column of the gas chromatograph is connected to a heated tube 44 that contains carbosieve material. The carbosieve materials is preferably activated at about 550° C. under helium before usage. The temperature of the carbosieve tube is fixed at about 540° C. during the experiments. The temperature of the carbosieve was controlled and monitored by a THERMOLYEN controller (model 12900) and with a LEYBOLD digital thermometer (model 666452) using K type thermal couples. Upon passing the heated carbosieve tube, oxygen will react with carbon to produce carbon oxides. The outlet of the carbosieve tube and a hydrogen gas 45 from the gas chromatograph are connected with a heated nickel catalyst tube 46. The nickel catalyst is maintained at a temperature of about 350° C. The catalyst could also be heated at about 400° C. With the help of a catalyst, carbon oxides react with hydrogen to give methane and water. The outlet of the nickel catalyst tube is then connected to the flame ionization detector 54 to analyze methane.

The detection limit of the flame ionization detector (10⁻¹² g/ml) limits the detection limit of this method i.e. low ppb. Hence, the oxygen gas is analyzed by a flame ionization detector. Thus, trace analysis of oxygen can be routine work if a gas chromatograph is equipped with a carbosieve tube and the nickel catalyst. Accordingly, this method is a very economic method for trace analyses of oxygen.

To verify the oxygen was the analyzed species and analysis of high purity oxygen where the flame ionization detector (FID peak) was very large, the temperature of the carbosieve material during the run was gradually reduced. The intensity of oxygen peaks continued to decrease until it disappeared because there was no conversion of oxygen to carbon oxides. Also, when air was analyzed a large peak could be seen at an oxygen retention time. Since air does not give FID peak under normal conditions, the detected PEAK must be due to the converted oxygen. When high purity nitrogen was analyzed no peaks were detected at the nitrogen expected retention times. However, a peak appeared at an oxygen retention time. This oxygen is usually present as a contaminant in a high purity nitrogen gas. In addition, to the confirmation of the supplier, the high purity of the nitrogen cylinder was confirmed by using the thermal conductivity detector (TCD) of the gas chromatograph.

The experimental procedure protecting the method follows:

An HP 5880 A gas chromatograph equipped with thermal conductivity detector (TCD), flame ionization detector (FID) and 5.0 ml-loop-6-port-V calco valve was used in all of the experiments. The carrier gas was helium. A column of 13 X molecular sieve 4′* ⅛″ in conjunction with carboxen 1004, 6′* 1-8″ mark has been used. The oven temperature was 90° C. Flow rate of helium was 20 ml/min. Injector temperature was 200° C. and the detectors' temperatures were 250° C. The pressure of H₂ and air for FID were 70 and 40 psi, respectively. The sample was introduced through a 1/16″ stainless steel line, see FIG. 3 (40) which is connected to the valve (42) to allow flushing of the sampling-loop and automatic injection of the sample.

The nickel catalyst was activated over night over hydrogen gas and 350° C. before use. The temperature of the catalyst was kept at 350° C. in the analysis. The temperature of the carbosieve was kept at 540° C.

### EXAMPLE 1

FIG. 4 shows the base line of the flame ionization detector without injection of any sample. The hump shown is due to by passing the oxygen trapper. FIG. 1 (50) in the carrier gas for three minutes. The oxygen in the carrier gas passed through the carbosieve tube and a nickel catalyst to give methane which is reflected as a three-minute hump in the gas chromatograph.

### EXAMPLE 2

Trace levels of oxygen in helium were prepared by passing high purity (99.999%) helium through fresh Hewlett Packard (PN 3150-0414) oxygen scrubber. This scrubber was designed to give less then 1 ppm oxygen in the mixture at a flow rate of 3 liters per minute. However, the flow rate used was 5 ml/min. This gave oxygen levels of much less then 1 ppm.

The outlet of the scrubber was connected to the sampling loop so that the flow rate was kept a 5 ml/min. About 5.0 minutes were allowed to flush the sampling loop before each injection.

Eleven samples were injected during a gas chromatograph run. The largest peaks were due to the oxygen in the outlet of the scrubber. Data acquisition and processing perimeters were:
Signal attenuation equals 2⁸, threshold equals 4, peak width equal 0.02 min., and chart speed was equal to 0.1 cm per minute. The sample were injected systematically every five minutes during the run which resulted in oxygen retention time of 7.50, 12.51, 17.52, etc. An oxygen concentration of 1 ppm gave gas chromatograph counts of 767 plus or minus 13 within an error of plus or minus 1.6%. When the same samples were analyzed under the same conditions using the thermal conductivity detector, nothing was shown in the base line.

### EXAMPLE 3

High purity 99.999% helium gas was analyzed without passing through an oxygen scrubber. No peaks were detected when the same gas was analyzed using the thermal conductivity detector of the gas chromatograph. Oxygen peaks resulted from periodic injections of high purity 99.999% helium samples. The large peaks and their retention times were due to the oxygen in the helium gas. The data acquisition perimeter were the same as that in example 2. The first two peaks were smaller then the other peaks, since the steady state of the system had not been reached. However, the rest of the eleven samples gave reproducible results with an error of plus or minus 1.5%.

### EXAMPLE 4

It was difficult to obtain a low ppb of oxygen gas as a standard. Many well known companies around the world apologized for not supplying any oxygen gas in the ppb range because they do not have equipment to measure such low levels of oxygen. The minimum concentration obtained was 520 ppb from the International Gasses and Chemicals Limited (INTERGAS), England it is an ISO 9002 company.

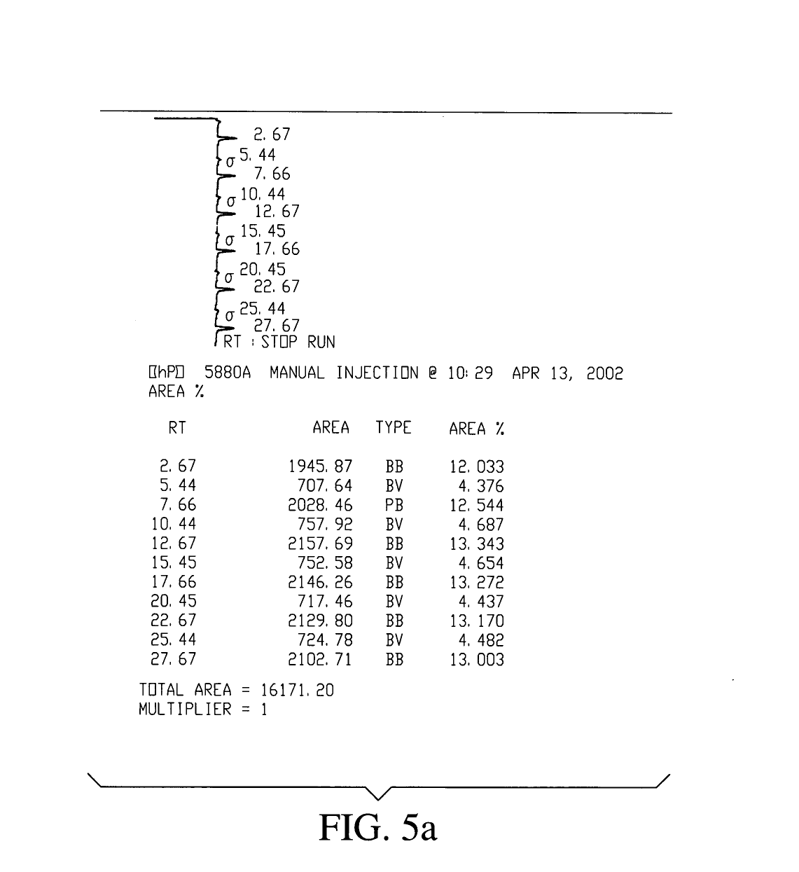

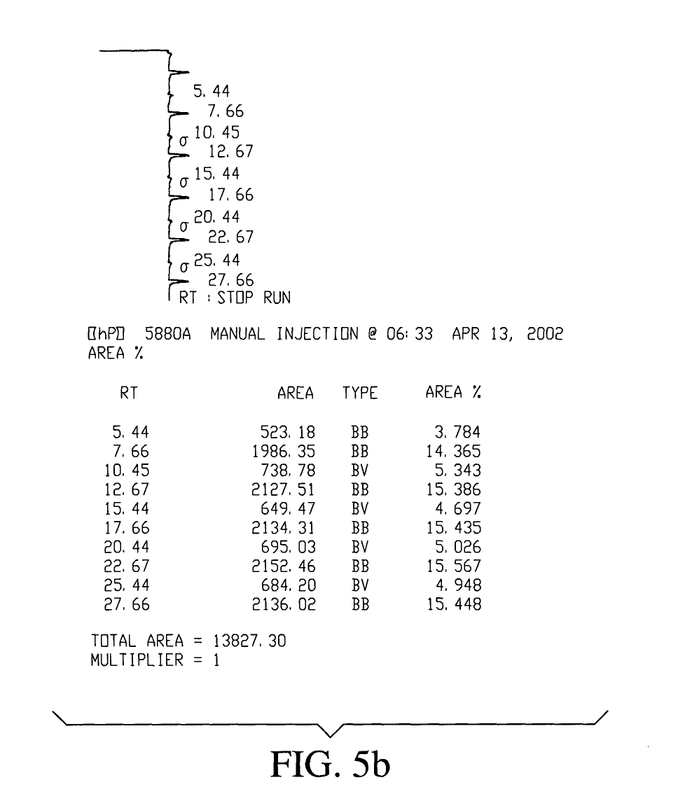

FIGS. 5a and 5b shows oxygen peaks resulting from periodic injection of 520 ppb of oxygen in helium balance. The attenuation of the GC signal equals 2⁸, the peak equals 0.02 min., threshold equals 4, and the chart speed was equal 0.2 cm per min. The average of the counts of the gas chromatograph in FIG. 5a was 2112 plus or minus 51 (plus or minus 2.4%) when 520 ppb was injected. The first sample is excluded from the average. FIG. 5b gave similar results with count numbers of 2107 plus or minus 68 (plus or minus 3.2%). Assuming exclusion of the first integrated sample in FIG. 5b, the area would be (plus or minus 0.5%).

In those cases where quantitative analysis is needed, an oxygen standard may be used. For example, for quantifying the analyzed oxygen one can use an internal standard method without the need of a calibration curve. However, FIGS. 5a and 5b illustrate one approach for calculating oxygen concentration. For example, FIG. 5b may be used as an example of calculating oxygen concentration based on FIG. 5a since 2,107 counts was obtained in FIG. 5b which indicates an oxygen concentration of 520 ppb based on the previous run in FIG. 5a which indicated 520 ppb by 2,112 counts.

An important result is that the gas chromatograph can easily detect compounds that produce 10 GC/counts. This means that one can easily detect very low ppb oxygen gas by using the current gas chromatographs. This provides, for the first time, the opportunity for the gas industry to analyze and prepare oxygen traces in the low ppb range using gas chromatographs.

While the invention has been described in connection with its preferred embodiments, it should be recognized the changes and modifications may be made therein without departing from the scope of the claims.

1. A method for analyzing trace amounts of oxygen in a relatively pure gas comprising the steps of:
separating the oxygen from a gas mixture;
converting the separated oxygen gas to a carbon oxide or oxides by passing the oxygen over carbon at an elevated temperature;
converting the carbon oxides to methane by mixing the carbon oxides with hydrogen with a heated nickel catalyst;
introducing the methane into a flame ionization detector to determine the amount of methane; and
comparing the amount of methane with an amount of methane produced in a control sample of a gas mixture with a known amount of oxygen.

2. A method for analyzing trace amounts of oxygen in a relatively pure gas comprising the steps of:
separating the oxygen from a gas mixture;
converting the separated oxygen gas to a carbon oxide or oxides by passing the oxygen over carbon at an elevated temperature;
converting the carbon oxides to methane by mixing the carbon oxides with hydrogen with a heated nickel catalyst;
introducing the methane into a flame ionization detector to determine the amount of methane; and
correlating the amount of oxygen required to produce the amount of methane indicated.

3. A method for measuring ultra low trace amounts of oxygen in a gas mixture comprising the steps of:
a). providing a gas chromatograph, a flame ionization detector, a mass of heated carbon and a heated nickel catalyst;
b). introducing a gas mixture to be analyzed and a carrier gas into the gas chromatograph to form a mixture of a gas mixture and carrier gas;
c). passing the mix of the gas mixture and carrier gas through a column of a gas chromatograph to thereby separate oxygen from the other gasses.
d). passing the oxygen through the heated carbon at an elevated temperature to thereby form carbon oxides;
e). providing a mass of hydrogen gas;
f). passing the carbon oxides over the heated nickel catalyst in the presence of hydrogen gas as to form methane; and
g). introducing the methane into the flame ionization detector to provide an indication of the amount of oxygen in the original gas mixture.

4. A method for measuring ultra low trace amounts of oxygen according to claim 2 in which said carbon is heated to a temperature of about 490° C. and 550° C.

5. A method for measuring ultra low trace amounts of oxygen according to claim 2 in which said carbon is heated to a temperature of about 550° C.

6. A method for measuring ultra low trace amounts of oxygen according to claim 4 in which said nickel catalyst is heated to a temperature of about 400° C.

7. A method for measuring ultra low trace amounts of oxygen according to claim 4 in which said nickel catalyst is heated to a temperature of about 350° C.

8. A method for measuring ultra low trace amounts of oxygen according to claim 3 in which the carrier gas is helium.

9. A method for measuring ultra low trace amounts of oxygen according to claim 3 in which the gas chromatograph is maintained at a temperature of about 90° C.
